(19) United States
(12) Patent Application Publication
(10) Pub. No.: US 2014/0323984 A1
(43) Pub. Date: Oct. 30, 2014

(54) Hand-Actuated Fluid Delivery Device Having Triggered Lock for Selectively Limiting Automatic Aspiration Stroke

(71) Applicant: Mallinckrodt LLC, Harzelwood, MO (US)

(72) Inventors: John K. Bruce, Burlington, KY (US); Geoffrey S. Strobl, Williamsburg, OH (US); David W. Wilson, Loveland, OH (US)

(21) Appl. No.: 14/358,061

(22) PCT Filed: Nov. 20, 2012
(86) PCT No.: PCT/US12/65980

§ 371 Date: May 14, 2014

Related U.S. Application Data
(60) Provisional application No. 61/562,547, filed on Nov. 22, 2011.

(51) Int. Cl. A61M 5/315

(52) U.S. Cl. CPC A61M 5/31581
USPC 604/223

(57) ABSTRACT

A hand-actuated injector (10) includes a handle assembly (200) for providing discharge and aspiration strokes for a plunger (108). The handle assembly (200) includes first and second scissor-like handle members (204, 212) that provide a discharge stroke for the plunger (108) when manipulated in a first manner, and an aspiration stroke for the plunger (108) when manipulated in an opposite, second manner. The handle assembly (200) also includes a meshed-gear locking mechanism or hinge (248) that selectively prevents or at least limits relative movement between the first and second handle members (204, 212) in a manner that is associated with providing an aspiration stroke for the plunger (108).

### CROSS-REFERENCE TO RELATED APPLICATIONS

This patent application is a non-provisional patent application of, and claims priority to, pending U.S. Provisional Patent Application Ser. No. 61/562,547, that is entitled “HAND-ACTUATED FLUID DELIVERY DEVICE HAVING TRIGGERED LOCK FOR SELECTIVELY LIMITING AUTOMATIC ASPIRATION STROKE,” that was filed on 22 Nov. 2011, and the entire disclosure of which is hereby incorporated by reference in its entirety herein.

### FIELD OF THE INVENTION

The present invention generally relates to the field of fluid delivery devices such as hand-held or hand-actuated injectors and, more particularly, to hand-actuated injectors that can be manipulated to limit an otherwise automatic aspiration stroke of an associated syringe.

### BACKGROUND

A “fluid delivery device” generally refers to an automatically or manually operated apparatus that facilitates the discharge of fluids from a syringe. Fluid delivery devices have been used to inject liquids such as contrast media and medicines into patients. Many fluid delivery devices allow a doctor or other user to control the rate at which a fluid is injected into a patient's body.

One type of fluid delivery device is a hand-held or hand-actuated fluid injector including pivotally interconnected, scissors-like actuation handles. One handle is pivotally connected to a barrel of a syringe and the other handle is pivotally connected to a plunger of the syringe. In this regard, pivoting the handles towards and away from each other respectively causes a fluid discharge stroke and a fluid aspiration stroke of the plunger. Some hand-held injectors are spring-loaded to automatically cause a fluid aspiration stroke of the plunger (e.g., immediately after a user releases the actuation handles).

### SUMMARY

A first aspect of the present invention is embodied by a hand-held injector that includes a first handle pivotally coupled to a second handle. The first and second handles are configured to provide a discharge stroke when first free end portions of the first and second handles are moved towards each other, and an aspiration stroke when the first free end portions of the first and second handles are moved away from each other. The hand-held injector also includes a self-activating lock, along with a selectively operable actuator that is connected to one of the first and second handles. The self-activating lock is configured to limit the first free end portions of the first and second handles from being moved apart when engaged by the actuator. The actuator is operable to disengage the self-activating lock when the actuator is moved in a direction towards the other of the first and second handles.

A second aspect of the present invention is embodied by a hand-powered injector including a first handle pivotally connected to a second handle at a locking hinge. The locking hinge includes a stationary gear associated with the first handle and a triggered actuator associated with the second handle. The triggered actuator is operable to engage with the stationary gear to limit first free end portions of the first and second handles from moving apart when the triggered actuator is left in an initial position, and is operable to remain disengaged from the stationary gear to allow the first free end portions of the first and second handles to move apart when the triggered actuator is moved in substantial unison with the second handle.

A number of feature refinements and additional features are separately applicable to each of the first and second aspects of the present invention. These feature refinements and additional features may be used individually or in any combination. As such, each of the following features that will be discussed may be, but are not required to be, used with any other feature or combination of features of the first and second aspects. The following discussion is separately applicable to each of the first and second aspects, up to the start of the discussion of a third aspect of the present invention. Initially, each feature of the first aspect may be used by the second aspect, alone or in any combination, and vice versa.

Second end portions of the first and second handles may be pivotally connected in any appropriate manner to a barrel and a plunger of a syringe. In this regard, movement of gripping portions, adjacent the first free end portions of the first and second handles, toward one another may cause movement of an associated syringe plunger in a discharge direction, while movement of the first free end portions of the first and second handle members apart may cause movement of an associated syringe plunger in an aspiration direction.

Any appropriate biasing member or resilient element (e.g., torsion spring) may be incorporated into the injector for use in exerting a torque or biasing force against the first and second handles that may serve to spread the first and second handles apart after the first and second handles have been at least partially squeezed and in the absence of engagement of the actuator with the stationary gear of the lock. When the actuator and lock are already at least partially engaged with each other (e.g., via another biasing member or resilient element urging the actuator towards and into engagement with the stationary gear), the torque exerted by the torsion spring may be used to drive the actuator and lock into further engagement with each other to limit movement of gripping portions of the first and second handles away from each other.

The actuator may be operable to move from a first engaged position with the lock to a second engaged position (and/or additional engaged positions) with the lock, where the gripping portions of the first and second handles are disallowed from moving apart from each other (e.g., via the torsion spring) in any of the engaged positions. Furthermore, the actuator may be operable to move between the various engaged positions in conjunction or unison with movement of one of the handles (e.g., the second handle) relative to the other of the handles (e.g., the first handle). In this regard, a user may be able to squeeze the gripping portions of the handles toward one another to induce a discharge stroke of an associated syringe plunger, and then terminate the discharge stroke by releasing at least one of the handles, whereupon the actuator may automatically engage (or remain engaged) with the lock to disallow the gripping portions of the handles from spreading apart (which would provide a corresponding aspiration stroke of an associated syringe plunger).

The actuator and the lock may be operable to more tightly grip, dig into, or otherwise engage with each other after each successive time that the gripping portions of the first and second handles are squeezed and then released. For instance, as the torsion spring or other resilient element may store increasing amounts of potential energy as the gripping portions of the first and second handles are moved closer and closer together, the increasing amounts of potential energy may be used by the torsion spring to further urge the actuator (e.g., connected to the second handle) and lock (e.g., fixedly or non-movably connected to the first handle) into engagement with each other. In this regard, the increasing levels of potential energy stored by the torsion spring may be used to further engage the actuator and lock, and thereby counterbalance the increased torque that would otherwise be used to spread the gripping portions apart. The actuator may have any appropriate feature(s) (e.g., pawl, series of teeth) that is (are) adapted to ratchet over one or more corresponding features (e.g., series of teeth) of the lock as the gripping portions of the first and second handle members are squeezed or otherwise moved towards each other to cause a discharge stroke of an associated syringe plunger. The ratcheting action may provide a user tactile feedback that the actuator and lock are in at least partial engagement and may further engage to disallow spreading apart of the gripping portions (and a subsequent aspiration stroke of an associated syringe plunger) upon or after release of at least one of the gripping portions.

In one arrangement, the actuator may be manipulable away from the lock so that the gripping portions of the first and second handles may be moved towards and/or away from each other free of engagement between the actuator and the lock. For instance, the actuator may include a trigger that, when depressed, serves to move (e.g., pivot) the pawl or teeth of the actuator away from the teeth of the lock. Maintaining depression of the trigger while squeezing the gripping portions of the first and second handle members toward one another thus allows a user to achieve a substantially smooth squeezing motion of the gripping portions (e.g., free of ratcheting movement of the actuator over the lock). For instance, the trigger may protrude into a finger aperture on the second handle. In this regard, a user may insert a finger into the finger aperture and depress the trigger in a direction towards the first handle using substantially the same motion that is used to move or squeeze the second handle towards the first handle. Furthermore, maintaining depression of the trigger while releasing at least one of the gripping portions may allow the torsion spring or other biasing member to spread the gripping portions apart and cause an aspiration stroke of the plunger. In some variations, slight squeezing of the gripping portions toward one another may be required to release the actuator from the lock (e.g. to reduce the pressure between the actuator and lock, prior to moving the actuator to a disengaged position relative to the lock).

A third aspect of the present invention is directed to a method that includes first engaging first and second engagement members using a first force so that a plunger of a hand-actuated fluid delivery device is locked from movement relative to a barrel in a direction away from a discharge nozzle (e.g., movement of the plunger away from the discharge nozzle may be characterized as an aspiration direction). The method also includes discharging fluid from the hand-actuated fluid delivery device by moving the plunger relative to the barrel from a first location, where the first force engages the first and second engagement members in the first position, towards the discharge nozzle (e.g., movement of the plunger toward the discharge nozzle may be characterized as a discharge direction); and then terminating the discharging step at a second location of the plunger relative to the barrel. In response to or after the terminating step, the method includes second engaging the first and second engagement members using a second force so that the plunger is locked from movement relative to the barrel in the direction away from the discharge nozzle, wherein the second force is greater than the first force.

A number of feature refinements and additional features are separately applicable to the third aspect of the present invention. These feature refinements and additional features may be used individually or in any combination. As such, each of the following features that will be discussed may be, but are not required to be, used with any other feature or combination of features of the third aspect. The following discussion is separately applicable to the third aspect, up to the start of a discussion of a fourth aspect of the present invention.

The method may include moving the first engagement member relative to the second engagement member from a first position relative to the second engagement member to a second position relative to the second engagement member, where the first engaging step occurs at the first position and the second engaging step occurs at the second position. For instance, the first position between the first and second engagement members may be a first manner in which teeth or gears of the first engagement member interlock with teeth or gears of the second engagement member, and the second position may be a different, second manner in which teeth or gears of the first engagement member interlock with those of the second engagement member (e,g., where more teeth are interlocked in the second position than in the first position). Moving the engagement members between the first and second positions may occur substantially simultaneously with the discharging step (e.g., in conjunction with the squeezing of first and second pivotally connected handles or handle members together that are respectively pivotally interconnected to the barrel and plunger, respectively).

In one arrangement, moving the engagement members between the first and second positions may entail ratcheting the first engagement member along the second engagement member from the first position to the second position (e.g., by way of ratcheting at least one pawl or tooth of the first engagement member along a series of gears or teeth of the second engagement member). In another arrangement, the moving step may involve moving the first engagement member away from the second engagement member, then moving the first and second engagement members from the first position to the second position in a manner such that the first engagement member is at least substantially free of contact with the second engagement member, and then moving the first engagement member back into engagement with the second engagement member. For instance, the first engagement member may be pivoted away from the second engagement member by way of depressing a trigger (e.g., in conjunction with squeezing the first and second handles together) to allow the first and second handles to be operated to provide a discharge stroke and without feeling the “ratcheting effect” of the first and second engagement members, and at the end of the desired discharge stroke the first engagement member may be moved back into engagement with the second engagement member (e.g., automatically by a biasing force). The first and second engaging steps may include biasing the first and second engagement members into engagement with each other using at least one biasing member (e.g., spring).

At least one biasing member may be used to automatically provide the force for moving the plunger in an aspiration direction (including where this biasing member provides the entirety of the force to move the plunger in an aspiration direction). Increasing amounts of potential energy may be stored in such a biasing member during the discharging step. This increased potential energy may be used to bias the engagement members into enhanced engagement with each other (e.g., via urging respective series of teeth of the first and second engagement members further into engagement with each other during the discharging step and as the potential energy in the noted biasing member progressively increases).

The method may also include disengaging the first and second engagement members and then aspirating fluid into the barrel of the hand-actuated fluid delivery device in response to the disengaging step. In one arrangement, the disengaging step may include moving (e.g., pivoting) one of the first and second engagement members away from the other of the first and second engagement members (e.g., by depressing a triggered actuator). In one variation, the disengaging step may also include first moving at least one of first and second handles members of the hand-actuated fluid delivery device towards the other of the first and second handle members (e.g., to reduce the pressure between the first and second engagement members).

A fourth aspect of the present invention is directed to a hand-actuated fluid delivery device that includes a barrel, a plunger, and a handle assembly. The handle assembly is interconnected with and operable to move the plunger relative to the barrel in both a discharge direction and an aspiration direction. The handle assembly includes first and second handle members that are pivotally interconnected with each other about a first axis at an intermediate location along the length of each of the first and second handle members. Moving free ends or free end portions of the first and second handle members at least generally toward each other (by a pivoting motion about the first axis) moves the plunger in a discharge direction, while moving free ends or free end portions of the first and second handle members at least generally away from each other (by a pivoting motion about the first axis) moves the plunger in an aspiration direction.

The handle assembly of the fourth aspect further includes a plunger lock that is associated with the first handle member. The actuator is pivotally connected with the second handle member about a second axis that is spaced from the first axis (the first axis again being that about which the first and second handle members move relative to one another). The first pivot axis and the second pivot axis may be at least substantially parallel with one another. In any case, the actuator is pivotable about the second axis into an engaged position with the plunger lock to limit the ability of the free ends or free end portions of the first and second handle members to be moved apart to in turn move the plunger in an aspiration direction. The actuator is also manually pivotable by a user about the second axis into a disengaged position relative to the plunger lock to allow the free ends or free end portions of the first and second handle members to be moved apart to in turn move the plunger in an aspiration direction.

A number of feature refinements and additional features are separately applicable to the fourth aspect of the present invention. These feature refinements and additional features may be used individually or in any combination. As such, each of the following features that will be discussed may be, but are not required to be, used with any other feature or combination of features of the fourth aspect. The following discussion is applicable to at least the fourth aspect.

The handle assembly may include at least one biasing member to bias the free ends or free end portions of the first and second handle members apart. Such a biasing member may be characterized as automatically providing an aspiration stroke for the hand-actuated fluid delivery device. The hand-actuated fluid delivery device could also be configured to require that a user provide the entirety of the forces to move the plunger in an aspiration direction (e.g., a user may be required to manually spread the free ends or free end portions of the first and second handle members apart to move the plunger in an aspiration direction). A combination of biasing forces and user-applied forces may be used to move the plunger in an aspiration direction.

The plunger lock and the actuator may be collectively characterized as a ratchet (e.g., the plunger lock being in the form of a gear and the actuator being in the form of a pawl). In any case, the plunger lock may be mounted to the first handle member and may be maintained in a fixed position relative to the first handle member. That is, although the plunger lock may move relative to the second handle member as the first handle member is moved, the plunger lock may be integrated with the first handle member such that there is no relative movement therebetween during any movement of the first handle member. In one embodiment, the plunger lock is in the form of a gear having a plurality of teeth that are disposed on at least a portion of the perimeter of this gear. The plunger lock may be characterized as being in the form of a stationary gear (e.g., stationary relative to the first handle member).

The second handle member may move counterclockwise about the first pivot axis (the axis about which second handle member moves relative to the first handle member) when the first and second handle members are operated to move the plunger in a discharge direction and in a first side view of the hand-actuated fluid delivery device. The actuator may move counterclockwise about the second axis (the axis about which the actuator is movable relative to the second handle member) when the actuator is moved from an engaged position with the plunger lock to a disengaged position relative to the plunger lock, and in the same first side view of the hand-actuated fluid delivery device. In one embodiment, the actuator is biased into its engaged position with the plunger lock (e.g., using one or more biasing members of any appropriate type and disposed in any appropriate arrangement).

The actuator may be moved separately or may be independently actuated compared to the operation of the first and second handle members to move the plunger in either a discharge direction or aspiration direction. In one embodiment, the actuator is automatically maintained in its engaged position with the plunger lock as the first and second handle members are operated to move the plunger in a discharge direction. As such, the interaction between the actuator and the plunger lock should limit the ability of the plunger to at least at some point in time move in the aspiration direction upon termination of the operation of the first and second handle members in a manner that would move the plunger in the discharge direction.

The actuator may be incorporated by the handle assembly such that it may be moved into a disengaged position relative to the plunger lock prior to and/or during operation of the first and second handle members in a manner that moves the plunger in a discharge direction. This may provide a “smoother feel” to an operator of the hand-actuated fluid delivery device during a discharge operation. In one embodiment, a common actuation or actuation motion is used to both move the actuator from an engaged position with the plunger lock to a disengaged position relative to the plunger lock, and to operate the first and second handle members in a manner that moves the plunger in a discharge direction. The actuator may at any time be returned to the engaged position with the plunger lock to limit the ability of the plunger to move in the aspiration direction. This return of the actuator to the engaged position with the plunger lock may be automatically provided by a biasing force exerted on the actuator, by a force manually applied by the user to the actuator, or a combination thereof. A number of feature refinements and additional features are separately applicable to each of above-noted first, second, third, and fourth aspects of the present invention. These feature refinements and additional features may be used individually or in any combination in relation to each of the above-noted first, second, third, and fourth aspects. Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that hand-powered injector includes “a resilient element” alone does not mean that the hand-powered injector includes only a single resilient element). Moreover, any failure to use phrases such as “at least one” also does not limit the corresponding feature to the singular (e.g., indicating that a hand-powered injector includes “a resilient element” alone does not mean that the hand-powered injector includes only a single resilient element). Use of the phrase “at least generally,” “at least substantially,” or the like in relation to a particular feature encompasses the corresponding characteristic and insubstantial variations thereof (e.g., indicating that a pivot axis is at least generally parallel to another pivot axis encompasses the pivot axes being parallel). Finally, a reference of a feature in conjunction with the phrase “in one embodiment” does not limit the use of the feature to a single embodiment.

### BRIEF DESCRIPTION OF THE FIGURES

FIG. 1 is a side view of one embodiment of a hand-held, hand-actuated fluid delivery device illustrating a lock and an actuator that may be used to engage or disengage with the lock to selectively inhibit an aspiration stroke of an associated plunger.

FIG. 2 is an exploded, perspective view of a handle assembly used by the hand-actuated fluid delivery device of FIG. 1.

FIG. 3 presents one embodiment of a method of using the hand-actuated fluid delivery device of FIG. 1.

FIG. 4 is a perspective view of the handle assembly of the hand-actuated fluid delivery device of FIG. 1, and illustrating first and second handle members of the fluid delivery device in a spread-apart, initial or home position.

FIG. 5 is a perspective view similar to that in FIG. 4, but illustrating the first and second handle members having been moved toward one another for a discharge stroke and with the actuator being engaged with the lock.

FIG. 6 is a perspective view similar to that in FIG. 4, but illustrating the first and second handle members having been moved toward one another for a discharge stroke and with the actuator now being disengaged from the lock.

### DETAILED DESCRIPTION

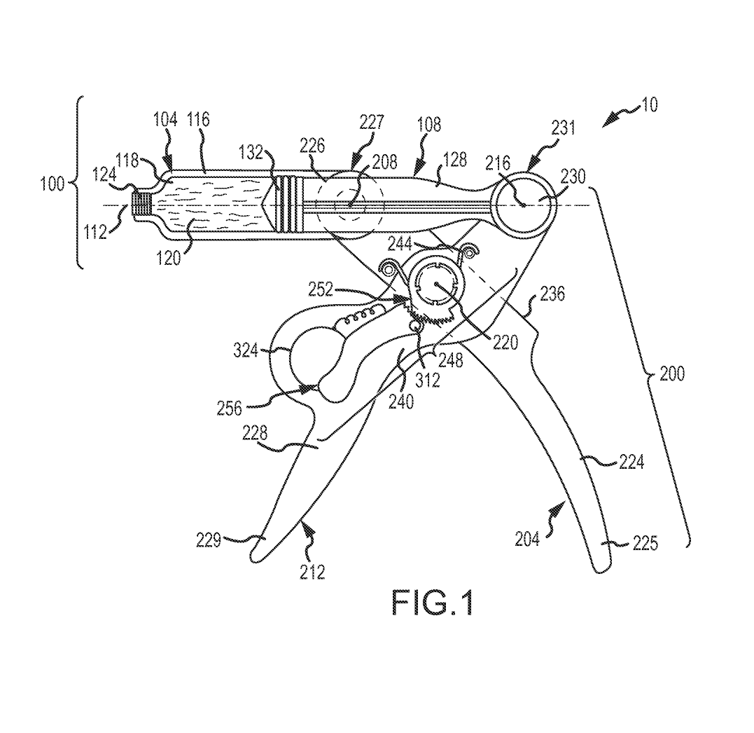

FIG. 1 is a side view of one embodiment of a hand-held, hand-actuated (e.g., hand powered) fluid delivery device or injector 10 that may be used by medical personnel or other users to manually inject one or more fluids into a patient. The hand-held injector 10 includes a syringe 100 having a barrel 104 and a piston or plunger 108, along with a handle assembly 200 that may be used to axially reciprocate or otherwise move the plunger 108 relative to the barrel 104 along a longitudinal axis 112 of the syringe 100. Generally, the handle assembly 200 is sized so as to be engaged and operated by a single hand of a user.

The barrel 104 of the hand-held injector 10 generally includes a body 116, a cavity 118 within the body 116 for holding at least one fluid 120 (e.g., a medical fluid), and a tip or discharge nozzle 124 for allowing the passage of fluid 120 therethrough (either into or out of the cavity 118 of the barrel 104). The plunger 108 generally includes a body 128 and a head 132 connected to or formed as one-piece with the body 128 for use in displacing fluid 120 through the discharge nozzle 124, and with the head 132 being positioned within the barrel 104. As used herein, a “discharge” stroke or direction corresponds with the head 132 moving within the cavity 118 along longitudinal axis 112 and towards tip 124 to discharge fluid 120 from the cavity 118, whereas an “aspiration” stroke or direction corresponds with the head 132 moving within cavity 118 along longitudinal axis 112 and away from discharge nozzle 124, for instance to draw fluid 120 (and/or one or more other fluids) into the cavity 118 or to at least allow one or more fluids to be loaded into the barrel 104.

The handle assembly 200 includes a first handle or first handle member 204 (e.g., a rear handle member) that is operable to be pivotally connected to the barrel 104 at a first pivot axis 208, along with a second handle or second handle member 212 (e.g., a front handle member) that is operable to be pivotally connected to the plunger 108 at a second pivot axis 216. The handle assembly 200 is sized such that the first handle member 204 and the second handle member 212 may be engaged by a common, single hand of a user. That is and for the condition shown in FIG. 1, one portion of a users hand may engage the first handle member 204, and another portion of this same hand may engage the second handle member 212 to manually actuate the injector 10 to discharge fluid 120 therefrom.

The first handle member 204 may include a gripping portion 224 generally adjacent a first free end portion 225 of the first handle member 204, and a barrel connection portion 226 generally adjacent a second end portion 227 of the first handle member 204. Similarly, the second handle member 212 may include a gripping portion 228 generally adjacent a first free end portion 229 of the second handle member 212, and a plunger connection portion 230 generally adjacent a second end portion 231 of the second handle member 212. Corresponding apertures (not shown/labeled) may be formed through the barrel connection portion 226 and the barrel 104 (and/or any appropriate bracket(s) associated with the barrel 104, not shown) and any appropriate pivot mechanism (e.g., pin, hinge, not shown/labeled) may be inserted through the corresponding apertures to allow for pivotal movement of the first handle member 204 relative to the barrel 104. As another example, a pin or hinge element on one of the barrel connection portion 226 or barrel 104 may be inserted through a corresponding aperture on the other of the barrel connection portion 226 or barrel 104. A similar arrangement may be used to form the pivotal connection between the plunger connection portion 230 and the plunger 108. In some arrangements, the barrel connection portion 226 and plunger connection portion 230 may be selectively releasable from the corresponding barrel 104 and plunger 108 to allow the handle assembly 200 to be utilized with a plurality of different syringes 100 (e.g., each syringe 100 could be in the form of a disposable). Any appropriate way for pivotally connecting the first handle member 204 to the barrel 104 and the second handle member 212 to the plunger 108 may be utilized.

With continued reference to FIG. 1, the first and second handle members 204, 212 are pivotally connected to each other at a third pivot axis 220 to allow for scissors-like, pivotal movement of the first and second handle members 204, 212 towards and away from each other. More specifically, the first handle member 204 includes a second handle member connection portion 236 located between its first and second end portions 225, 227, and the second handle member 212 includes a first handle member connection portion 240 located between its first and second end portions 229, 231. The second handle member connection portion 236 of the first handle member 204 is pivotally interconnected to the first handle member connection portion 240 of the second handle member 212 to allow the scissors-like movement of the first and second handle members 204, 212. In this regard, moving the gripping portion 224 or first free end portion 225 of the first handle member 204 at least generally towards the gripping portion 228 or first free end portion 229 of the second handle member 212 provides a fluid discharge stroke of the plunger 108 (e.g., by decreasing the spacing between the barrel connection portion 226 and the plunger connection portion 230), while moving the gripping portions 224, 228 or first free end portions 225, 229 at least generally away from each other provides an aspiration stroke of the plunger 108 (e.g., by increasing the spacing between the barrel connection portion 226 and the plunger connection portion 230).

The handle assembly 200 may also include at least one biasing member or resilient element 244 (e.g., torsion or other type of spring) that is appropriately interconnected to the first and second handle members 204, 212, such as adjacent to and/or about the third pivot axis 220. The resilient element 244 is operable to exert a biasing force or torque on the first and/or second handle member 204, 212 in any appropriate manner for use in causing the handle members 204, 212 to spread apart and correspondingly provide an (automatic) aspiration stroke of the plunger 108. That is, the resilient element 244 biases the gripping portions 224, 228 of the handle members 204, 212 away from each other (e.g., to the spaced apart configuration shown in FIG. 1). More specifically, upon a user squeezing gripping portions 224, 228 towards each other (i.e., compressing the gripping portions 224, 228 together) to provide a discharge stroke of the plunger 108 and thereafter releasing at least one of the gripping portions 224, 228, the biasing force or torque exerted by the resilient element 244 may in at least some situations operate to push the gripping portions 224, 228 away from each other and simultaneously provide the aspiration stroke of the plunger 108.

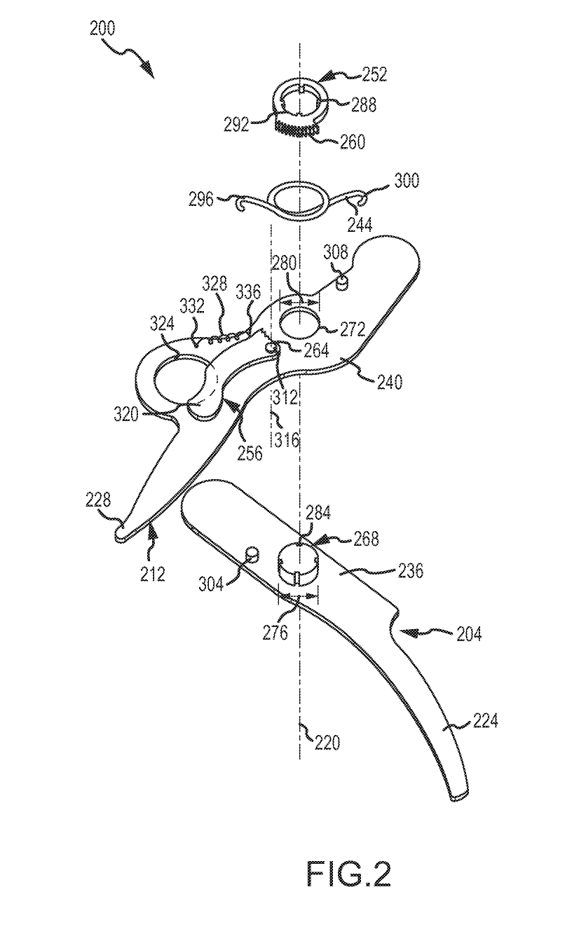

While the automatic aspiration stroke provided by the resilient element 244 may be desired in at least some circumstances, medical personnel or other users sometimes encounter other situations where it would be desirable to selectively prevent or at least limit the above-discussed automatic aspiration stroke of a syringe. In this regard, the handle assembly 200 includes what may be characterized as a locking hinge 248 that is broadly operable to pivotally interconnect the first and second handle members 204, 212 at the third pivot axis 220, as well as allow a user to selectively allow or limit an aspiration stroke of a syringe connected to the handle assembly 200, such as syringe 100. Referring now to both FIGS. 1 and 2, the locking hinge 248 generally includes a pivot member 268 that pivotally connects the first handle member 204 to the second handle member 212, a first engagement member such as a self-activating lock 252 associated with one of the first and second handle members 204, 212, and an opposed engagement member such as a selectively operable or triggered actuator 256 associated with the other of the first and second handle members 204, 212.

The lock 252 and the actuator 256 may be collectively characterized as a “ratchet.” For instance, the lock 252 may be characterized as the gear or rack (having a series or a plurality of teeth that are spaced from one another), and the actuator 256 may be characterized as the pawl (e.g., including or in the form of at least one tooth, finger, projection, or the like that may be disposed in the space between an adjacent pair of teeth 260 of the lock 252). In the illustrated embodiment, the lock 252 is associated and moves along with the first handle member 204, while the actuator 256 is mounted on and may move along with the second handle member 212 (the actuator 256 also being selectively movable relative to the second handle member 212, as will be discussed below). When the actuator 256 is appropriately engaged with the lock 252 and as shown in FIG. 1: 1) the actuator 256 may move relative to the lock 252 in one direction by moving the gripping portions 224, 228 of the handle members 204, 212 at least generally toward one another to move the plunger 108 in the discharge direction (e.g., the actuator 256 may be characterized as moving counterclockwise at least generally about the lock 252 in the view presented in FIG. 1 by a pivoting joint 312 (between the actuator 256 and second handle member 212) moving counterclockwise about a pivot 268 (fixed to the first handle member 204)); but 2) movement of the actuator 256 relative to the lock 252 in the opposite direction may be limited—once at least one tooth 264 of the actuator 256 is disposed between an adjacent pair of teeth 260 of the lock 252, the actuator 256 may be characterized as being unable to move clockwise relative to and about the lock 252 in the view presented in FIG. 1 by precluding the pivoting joint 312 (between the actuator 256 and second handle member 212) from moving clockwise at least generally about the pivot 268 (fixed to the first handle member 204)), for instance in response to and/or when attempting to move the gripping portions 224, 228 of the handle members 204, 212 at least generally away from one another and which would move the plunger 108 in an aspiration direction.

FIG. 1 illustrates a home or initial position for the handle assembly 200—where the gripping portions 224, 228 of the handle members 204, 212 are at their maximum spacing. At this time and for the illustrated embodiment, the actuator 256 is engaged with the lock 252. However, the actuator 256 could be incorporated such that it was totally disengaged with the lock 252 in the noted home position for the handle assembly 200, but would move into the engaged position with the lock 252 at some point in time during operation of the handle assembly 200 in a manner that moves the plunger 108 in the discharge direction (e.g., shortly after the start of the movement of the plunger 108 in the discharge direction).

In one arrangement, the first and second handle members 204, 212 may each include first and second halves that are generally operable to mount about (e.g., sandwich) at least a portion of the locking hinge 248 and connect to each other. For instance, the second handle member 212 shown in FIG. 1 may represent a first half of the second handle member 212, and a substantially identical second half (not shown) of the second handle member 212 may be mounted over the locking hinge 248 and attached to the corresponding first half in any appropriate manner (e.g., via snap fittings, adhesives) to substantially conceal the locking hinge 248 (e.g., except for the portion of the actuator 256 designed to protrude into finger aperture 324). Also, the first handle member 204 shown in FIG. 1 may represent a first half of the first handle member 204, and a substantially identical second half (not shown) of the first handle member 204 may be mounted over the second half of the second handle member 212 and attached to the first half of the first handle member 204 in any appropriate manner. In any event, and as will be discussed more fully in the following discussion, the actuator 256 is designed to disallow or preclude an aspiration stroke of the plunger 108 within the barrel 104 when left in an engaged position with the lock 252, and to allow an aspiration stroke of the plunger 108 within the barrel 104 when appropriately manipulated or activated by a user into a disengaged position relative to the lock 252.

FIG. 2 presents an exploded, perspective view of the handle assembly 200, and illustrates the various components of the above-noted locking hinge 248. Components such as the syringe 100, barrel and plunger connection portions 226, 230 of the first and second handle members 204, 212, and the like have been removed for clarity. The pivot member 268 (e.g., a protrusion, nub, post) may be fixedly disposed on the first handle member 204 (i.e., it may be non-movable relative to the first handle member 204) in any appropriate manner and may be sized to be received by an aperture 272 in the first handle member connection portion 240 of the second handle member 212. More specifically, a diameter 276 of the pivot member 268 could be slightly smaller than a diameter 280 of the aperture 272 in the second handle member 212 to allow the aperture 272 to be fit over the pivot member 268 in a manner that allows for pivoting of the second handle member 212 relative to the first handle member 204 about the pivot member 268.

The lock 252 for the locking hinge 248 may include any appropriate locking feature such a series of gears, cogs or teeth 260 (e.g., a plurality of spaced teeth 260 disposed on a perimeter of the lock 252), and may be fixedly connected to the first handle member 204 in any appropriate manner. That is, the lock 252 may be incorporated by the first handle member 204 in a manner such that it is at least substantially non-movable relative to the first handle member 204. In this regard, the lock 252 may be referred to as a stationary gear (i.e., stationary relative to the first handle member 204). However, the lock 252 may be characterized as moving along or in conjunction with the first handle member 204.

The teeth 260 of the lock 252 may be disposed equidistantly from the third pivot axis 220, which again coincides with the pivot member 268 on which the lock 252 is mounted. For instance, the plurality of teeth 260 may be characterized as collectively defining an arcuate segment on the perimeter of the lock 252, where this arcuate segment may be “centered” at the third pivot axis 220.

The actuator 256 may be positioned or otherwise oriented relative to the lock 252 so as to be engaged with the series of teeth 260 of the lock 252. More specifically, the actuator 256 may include any appropriate feature (such as a corresponding series of gears, cogs or teeth 264, although the actuator 256 could utilize a single gear tooth, finger, projection, or the like) that is operable to selectively ratchet along the series of teeth 260 of the lock 252 as the gripping portions 224, 228 of the first and second handle members 204, 212 are moved at least generally toward one another (or as at least one of the gripping portions 224, 228 is moved towards the other of the gripping portions 224, 228), but that interlocks with the series of teeth 260 of the lock 252 so as to prevent or at least limit movement of the gripping portions 224, 228 away from each other when at least one of the gripping portions 224, 228 is released (as well as when the actuation force, that is used to force the gripping portions 224, 228 toward one another, is at least reduced, in which case the biasing force provided by the biasing element 244 may attempt to move the gripping portions 224, 228 apart). It should be noted that the actual number and shape of teeth in the series of teeth 260 (lock 252) and the series of teeth 264 (actuator 256) need not necessarily be the same. Rather, the series of teeth 264 (or other locking feature(s)) of the actuator 256 and the series of teeth 260 (or other locking feature(s)) of the lock 252 need only be capable of allowing ratcheting of the at least one tooth 264 of the actuator 256 along the series of teeth 260 of the lock 252 when the gripping portions 224, 228 are moved toward one another, and generally disallowing movement of at least one tooth 264 of the actuator 256 relative to the series of teeth 260 of the lock 252 when an attempt is made to move the gripping portions 224, 228 of the handle members 204, 212 away from each other to move the plunger 108 in an aspiration direction, such as in response to the biasing force provided by the resilient element 244 (e.g., that biases the gripping portions 224, 228 away from each other to provide an automated aspiration stroke for the injector 10). However, it should be appreciated that having multiple teeth 264 of the actuator 256 each being disposed between a different pair of teeth 260 of the lock 252 should provide more resistance to relative movement between the lock 252 and actuator 256 in the non-ratcheting direction (the direction coinciding with increasing the spacing between the gripping portions 224, 228 of the handle members 204, 212, that provides a corresponding increase in the spacing between the barrel connection portion 226 and the plunger connection portion 230, and moves the plunger 108 in an aspiration direction). The interaction between the actuator 256 and the lock 252 will be discussed in more detail below.

In one arrangement, the pivot member 268 may include one or more features that are adapted to engage with the lock 252 to prevent or at least limit rotational movement of the lock 252 relative to the first handle member 204. For instance, the pivot member 268 may include one or more openings 284 that are adapted to engage with one or more corresponding projections or other features on the lock 252 (such as splines 288) in a manner that prevents or at least limits rotation of the lock 252, about the pivot member 268 and relative to the first handle member 204, when the one or more splines 268 are engaged with the one or more openings 284. As shown, the pivot member 268 may include the one or more openings 284 located on an outer periphery thereof, while the lock 252 may include an aperture 292 with the one or more splines 288 located on an interior periphery thereof. Thus, each of the aperture 292 of the lock 252 and the aperture 272 of the second handle member 212 may receive the pivot member 168. However, the lock 252 is prevented or at least limited from rotating relative to the first handle member 204 owing to the spline(s) 288 (or other feature(s)) interacting with the aperture(s) 284 (or other feature(s)) of the pivot member 268, but the lock 252 again is allowed to move relative to the second handle member 212.

During assembly of the handle assembly 200, the second handle member 212 may be disposed over the first handle member 204 such that the pivot member 268 is inserted at least into (and possibly through) the aperture 272 of the second handle member 212, and then the resilient member 244 may be disposed over the pivot member 268. As part of this process, first and second ends or portions 296, 300 of the resilient member 244 may be respectively fixed or anchored to the first and second handle members 204, 212 in any appropriate manner so that when the resilient member 244 is torqued in a first rotational direction by way of the gripping portions 224, 228 of the first and second handle members 204, 212 being moved toward one another, the resilient member 244 is operable to exert an opposing biasing force or torque in an opposed second rotational direction that may function to spread the gripping portions 224, 228 of the first and second handle members 204, 212 apart. As just one example, the first and second handle members 204, 212 may include respective protrusions 304, 308 (e.g., posts, nubs) about which the first and second ends 296, 300 of the resilient member 244 can be respectively bent or otherwise manipulated. In other arrangements, the resilient member 244 may be disposed in other locations or orientations (e.g., between the first and second handle members 204, 212) so long as it exerts a biasing force or torque for use in urging gripping portions 224, 228 of the handle members 204, 212 away from each other (e.g., to bias the handle assembly 200 into a configuration that moves the plunger 108 in an aspiration direction).

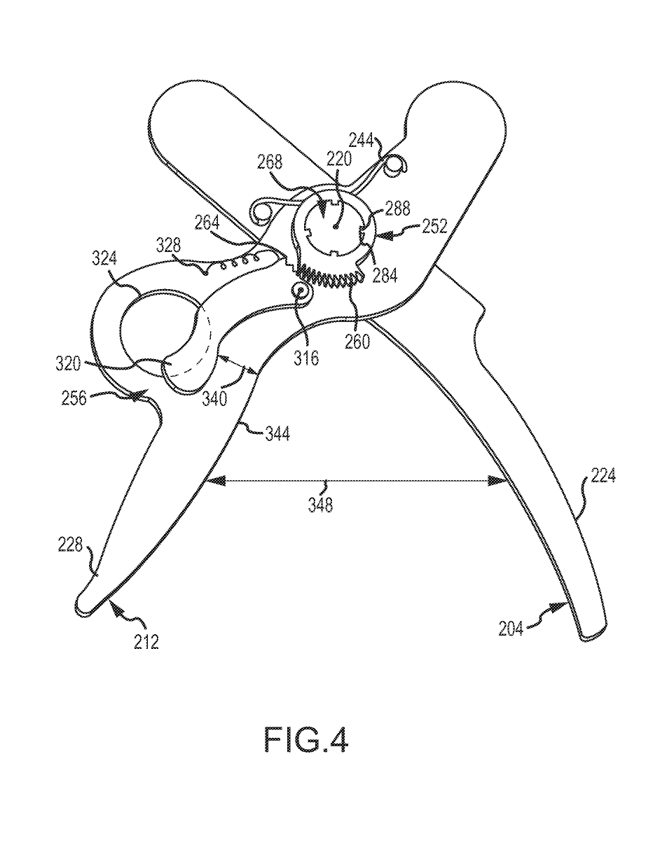

Assembly of the handle assembly 200 may also include disposing the aperture 292 of the lock 252 over the pivot member 268 such that the apertures 284 (or other rotation limiting features) of the pivot member 268 receive or otherwise engage with the splines 288 (or other corresponding rotation limiting features) of the lock 252. See FIGS. 4-6, discussed below. At this point, the first and second handle member 204, 212 are generally pivotal relative to each other, and the lock 252 is generally fixed relative to the first handle member 204 but rotatable relative to the second handle member 212 (i.e., the lock 252 moves along with the first handle member 204, and is movable relative to the second handle member 212). The lock 252 may also be substantially fixed against removal from the first and second handle members 204, 212. For instance, the splines 288 may be sized to be press-fit into apertures 284, and/or the lock 252 may be adhered to the pivot member 268. Other arrangements are envisioned and included within the scope of the present disclosure.

With reference now primarily to FIG. 2, the actuator 256 is pivotally connected to the second handle member 212 (e.g., at the first handle member connection portion 240) via a pivoting joint 312 (e.g., a pivot pin or other feature pivotally connecting the actuator 256 to the second handle member 212) so as to be pivotable or rotatable about a fourth pivot axis 316 that is substantially parallel to the first, second and third pivot axes 208, 216, 220. Also, the actuator 256 generally includes the series of teeth 264 disposed to one side of the fourth pivot axis 316 and a trigger or gripping portion 320 disposed to an opposed side of the fourth pivot axis 316 such that manipulation of the gripping portion 320 in one of first and second rotational directions generally induces a corresponding rotational or pivotal movement of the series of teeth 264 about the fourth pivot axis 316 via the pivoting joint 312.

One or more biasing members of any appropriate type, such as a resilient element 328 (e.g., coil or other type of spring), may be provided to maintain the actuator 256 in an initial, starting position such that the gripping portion 320 at least partially protrudes into a finger aperture 324 of the second handle member 212 (e.g., in the absence of any external force(s) being exerted on the actuator 256). More generally, one or more biasing elements may be used to maintain the actuator 256 in an engaged position relative to the lock 252 (e.g., when not being manually actuated or manipulated by a user). For instance, the resilient element 324 may have a first portion or end 332 fixedly attached or anchored to the second handle member 212 and a second portion or end 336 fixedly attached or anchored to a portion of the actuator 256 in any appropriate manner (e.g., molding, welding, adhesives). In this regard, movement of the actuator 256 in a counterclockwise direction in the view of FIG. 1 (about the pivoting joint 312) may apply a compressive load on the resilient element 328 which is stored as potential energy and which may be subsequently used by the resilient element 328 to bias the actuator 256 towards the lock 252. That is, the resilient member 328 may be characterized as biasing the actuator 256 into an engaged position with the lock 252.

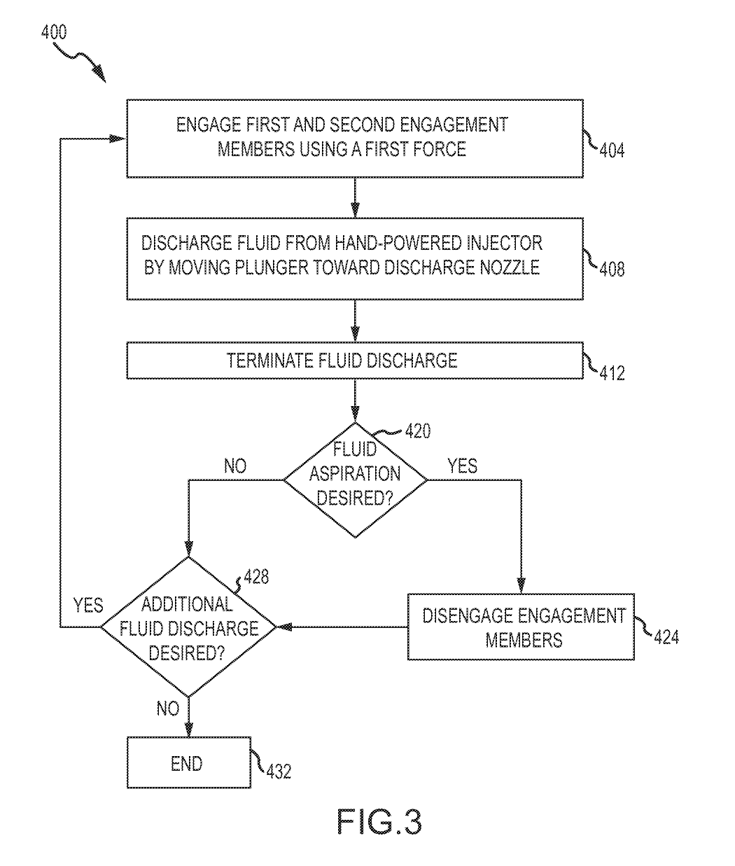

Turning now to FIG. 3, one embodiment of a method 400 of using the hand-held injector 10 will be discussed. In conjunction with discussion of the method 400, reference will also be made to FIGS. 1 and 4-6 which illustrate various operational stages of the handle assembly 200 (with the syringe 100 and other components having been removed for clarity from FIGS. 4-6). Turning initially to FIG. 4, an assembled perspective view of the handle assembly 200 is shown with each of the various components of the handle assembly 200 being in an initial or “home” position (e.g., the gripping portions 228, 224 of the handle members 212, 204 being biased apart to their maximum spacing by the resilient member 244). In one arrangement or configuration for the actuator 256, the series of teeth 264 of the actuator 256 may be completely disengaged from or otherwise separated from the series of teeth 260 of the lock 252 at this time and as previously noted. In another arrangement or configuration, and as shown in FIG. 4, one or more teeth 264 of the actuator 256 may be at least partially engaged with the series of teeth 260 of the lock 252 in the initial or home position for the handle assembly 200 so as to prevent or at least limit an attempt to move the gripping portions 224, 228 of the first and second handle members 204, 212 away from each other (e.g., via the biasing forces provided resilient element 244 in the situation where at least some potential energy is stored in the resilient element 244 at this time, although this is not a requirement), which would otherwise cause movement of the plunger 108 in an aspiration direction relative to the barrel 104 (e.g., via the head 132 of the plunger 108 moving away from the discharge nozzle 124). At least one of the teeth 264 of the actuator 256 may be engaged with a perimeter of the lock 252 having the teeth 260 (and may in fact be disposed between an adjacent pair of teeth 260 of the lock 252 at this time) to restrain relative movement between the lock 252 and the actuator 256 in the non-ratcheting direction (e.g., a relative movement of the actuator 256 in the clockwise direction about the lock 252 in the view shown in FIGS. 1 and 4).

As discussed previously, the series of teeth 260 of the lock 252 may be designed to allow ratcheting movement of the series of teeth 264 of the actuator 256 along the series of teeth 260 of the lock 252 in a first direction when the gripping portions 224, 228 of the first and second handle members 204, 212 are moved towards each other, and generally to disallow or preclude movement of the series of teeth 264 of the actuator 256 along the series of teeth 260 of the lock 252 in a second direction when an attempt is made to move the gripping portions 224, 228 away from each other (such as in response to the biasing force exerted on the handle members 204, 212 by the resilient element 244 that tends to increase the spacing between the gripping portions 224, 228, to in turn move the plunger 108 in an aspiration direction). For instance, each tooth 260 of the series of teeth 260 of the lock 252 may have first and second opposing surfaces, where the first surface has a more gradual incline than does the second surface. This arrangement generally allows the series of teeth 264 of the actuator 256 to ride over the first surfaces of the series of teeth 260 of the lock 252 when moved in the first direction (e.g., corresponding to compression of the gripping portions 224, 228 of the first and second handle members 204, 212), and to not ride over the second surfaces of the series of teeth 260 of the lock 252 when an attempt is made to move the series of teeth 264 of the actuator 256 in the second direction (e.g., corresponding to a spreading apart of the gripping portions 224, 228 of the handle members 204, 212).

The method 400 may include first engaging 404 first and second engagement members (e.g., actuator 256 and lock 252) so that the plunger 108 is locked from movement within the barrel 104 in a direction away from the discharge nozzle 124—the plunger 108 may be precluded from moving in the aspiration direction at this time. In the FIG. 4 configuration (again, the maximum spacing between the gripping portions 224, 228 of the handle members 204, 212, and where the actuator 256 is engaged with the lock 252), the resilient element 244 may be exerting a biasing force of a first magnitude on the handle members 204, 212 at this time and in a direction that tends to move the gripping portions 224, 228 apart. However, as the actuator 256 is engaged with the lock 252, this biasing force is unable to actually move the gripping portions 224, 228 further apart from the position shown in FIG. 4.

As shown in FIG. 4, the gripping portion 320 of the actuator 256 may be generally disposed a distance 340 from an edge 344 of the second handle member 212 (or other reference point/location on the second handle member 212), and the second handle member 212 (e.g., the gripping portion 228) may be disposed a distance 348 from the first handle member 204 (e.g., the gripping portion 224). Numerous other additional or alternative distances may be defined to illustrate the various relative positions between the actuator 256 and the first and second handle members 204, 212. Thus, it is to be understood that the distances 340, 348 (as well as other distances disclosed herein) have merely been provided to assist the reader in understanding the operation of the handle assembly 200.

The method 400 of FIG. 3 may also include discharging 408 fluid 120 from syringe 100 by moving the plunger 108 towards the discharge nozzle 124 (e.g., moving the plunger 108 in the discharge direction). For instance, the first and second handle members 204, 212 of the handle assembly 200 may be squeezed, compressed or otherwise moved toward one another (which decreases the spacing between the barrel connection portion 226 and plunger connection portion 23), to in turn move the plunger 108 in a discharge direction. This may be done in a manner that is free of depressing or otherwise manipulating the gripping portion 320 of the actuator 256 in a manner that would otherwise cause the actuator 256 to move about the fourth pivot axis 316 to disengage the actuator 256 from the lock 252. It should be noted that this particular operation of the handle assembly 200 may also increase the biasing forces being exerted by the resilient member 244 on the handle members 204, 212 (and which is again directed to increase the spacing between the gripping portions 224, 228 of the handle member 204, 212).

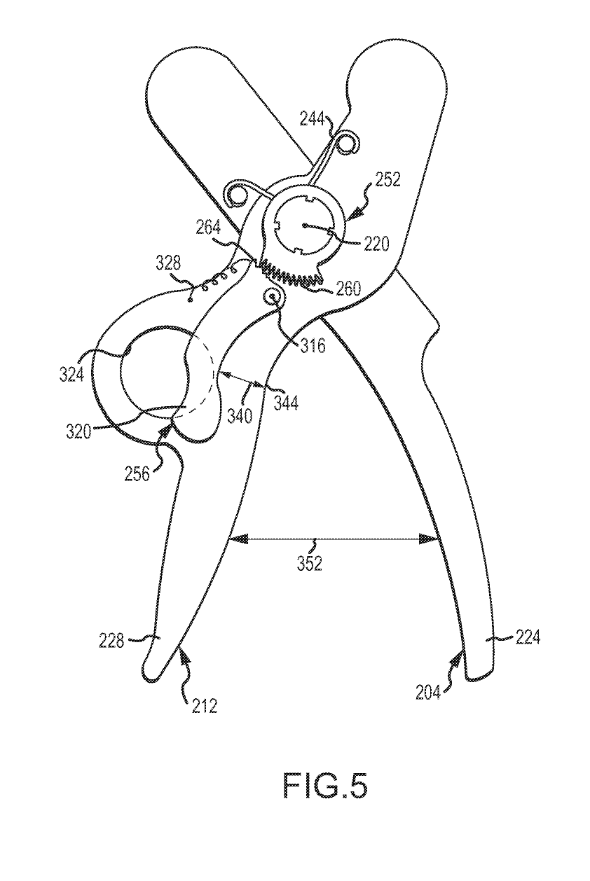

For the discharge step 408 of FIG. 3, the first and second handle members 204, 212 may be moved from the relative position between each other shown in FIG. 4, whereby the first and second handle members 204, 212 are separated by the distance 348, to the relative position between each other shown in FIG. 5, whereby the first and second handle members 204, 212 are now separated by a distance 252 that is less than the distance 348. The method 400 may include terminating 412 the discharging step 408 by way of, for instance, releasing at least one of the first and second gripping portions 224, 228 of the first and second handle members 204, 212 (which may or may not be accidental or unintentional), or more generally by reducing the actuation force being manually exerted by one hand of a user on one or more of the handle members 204, 212. This termination step 412 may be undertaken at any time between the relative position of the handle members 204, 212 presented in FIG. 4, and when the plunger 108 has reached the end of the barrel 104 (which could coincide with the gripping portions 224, 228 of the handle members 204, 212 being engaged with one another).

A number of observations can be made by comparing the illustration of the handle assembly 200 in FIG. 4 to that in FIG. 5. First, the gripping portions 224, 228 of the first and second handle members 204, 212 have been moved toward one another, which has induced a corresponding movement of the barrel and plunger connection portions 226, 230 of the handle members 204, 212 toward one another via the third pivot axis 220, and which then moves the head 132 of the plunger 108 in a discharge direction from a first position within the barrel 104 to a second position within the barrel 104 that is closer to the discharge nozzle 124 than is the first position. Also, the fourth pivot axis 316 has partially revolved about the third pivot axis 220 (e.g., the pivoting joint 312 (between the actuator 256 and the second handle member 212) has pivoted about the pivot member 268 (anchored relative to the first handle member 204)). Additionally, the resilient member 328 has assisted in maintaining the gripping portion 320 of the actuator 256 generally at substantially the distance 340 from the edge 344 of the second handle member 212 so that the actuator 256 continues to engage with the lock 252 (e.g. to restrain relative movement between the actuator 256 and the lock 252 in the non-ratcheting direction). That is, the resilient member 328 maintains the actuator 256 in engagement with the lock 242. Although the resilient member 328 biases the actuator 256 into engagement with the lock 252, it should be appreciated that it may also accommodate a small angular oscillatory motion of the actuator 256 about the pivoting joint 312 as the actuator 256 moves relative to the lock 252 in the ratcheting direction.

Upon termination 412 of the discharge step 408, and with the actuator 256 remaining engaged with the lock 252, the plunger 108 should be unable to undertake a complete aspiration stroke (although the plunger 108 could in fact move a small distance in the aspiration direction until at least one tooth 264 of the actuator 256 is seated in the spacing between an adjacent pair of teeth 260 of the lock 252, at which time further movement of the plunger 108 in the aspiration direction should be terminated). Neither the biasing forces being provided by the resilient member 244, nor forces being manually exerted by a user on the handle assembly 200, should be able to increase the spacing between the gripping portions 224, 228 of the handle members 204, 212 at this time and move the plunger 108 further in the aspiration direction. That is and in some situations, the plunger 108 may not be locked from movement away from the discharge nozzle 124 immediately after or simultaneous with the terminating step 412. For instance, in the situation where a tooth of the series of teeth 264 of the actuator 256 has not yet landed in the space (not labeled) between an adjacent pair of teeth of the series of teeth 260 of the lock 252, the resilient element 244 may function to spread apart the gripping portions 224, 228 of the first and second handle members 204, 212 by a small distance (and thus cause a small, initial movement of the plunger 108 in the aspiration direction) until the above-mentioned tooth of the actuator 256 has landed between the space between the above-mentioned adjacent pair of teeth of the lock 252.

As noted above, operating the handle assembly 200 to move the plunger 108 in the discharge direction may also increase the biasing forces being exerted by the resilient member 244 on the handle members 204, 212 (and which is again directed to increasing the spacing between the gripping portions 224, 228 of the handle members 204, 212). As such, the biasing force being exerted by the resilient member 244 is of a second magnitude in the FIG. 5 configuration of the handle assembly 200, which is larger than the first magnitude of biasing force being exerted by the resilient member 244 in the FIG. 4 configuration of the handle assembly 200 and noted above. This increased biasing force, or a force being manually exerted by a user on the handle assembly 200 in an attempt to increase the spacing between the gripping portions 224, 228 of the handle members 204, 212, at a time when the actuator 256 remains engaged with the lock 252, actually increases the resistance provided by the actuator 256 and lock 252 to movement of the plunger 108 in the aspiration direction. For instance, the teeth 264 of the actuator 256 and/or the teeth 260 of the lock 252 may be orientated such that these forces (singularly or in combination) would tend to attempt to move the actuator 256 in a clockwise direction about the pivoting joint 312 (between the actuator 256 and the second handle member 212), which forces the actuator 256 against the lock 252. This may provide further resistance to movement of the plunger 108 in the aspiration direction.

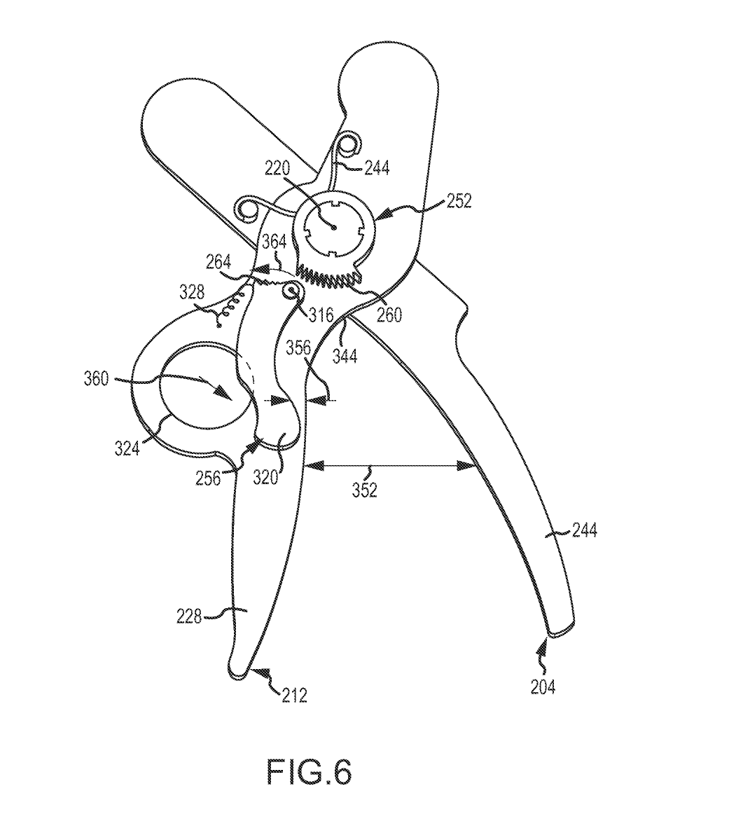

Turning back to FIG. 3, the method 400 may include determining 420 whether fluid aspiration (e.g., an aspiration stroke of the plunger 108, or movement of the plunger 108 in an aspiration direction) is desired. In response to a positive determination at step 420, the method 400 may include disengaging the actuator 256 from the lock 252 (step 424) to allow the torque or biasing force being exerted by the resilient member 244 to increase the spacing between the gripping portions 224, 228 of the first and second handle members 204, 212. With reference now to FIGS. 5 and 6, a user may squeeze or move the gripping portion 320 of the actuator 256 (e.g., via inserting a finger through the finger aperture 324) in a direction 360 towards the first handle member 204, or otherwise move the actuator 256 in a counterclockwise direction 364 about the fourth pivot axis 316 in the view shown in FIG. 1, to clear the series of teeth 264 of the actuator 256 away from the series of teeth 260 of the lock 252. The user may move the actuator 256 about the fourth pivot axis 316 so that a distance 356 (see FIG. 5) between the actuator 256 and the edge of the second handle member 212 becomes smaller than the distance 340 (see FIGS. 4-5). In one arrangement, the user may be required to both depress the gripping portion 320 of the actuator 256 and intentionally manipulate the gripping portions 224, 228 of the first and second handle members 204, 212 towards from each other (e.g., via slightly squeezing the gripping portions 224, 228) to allow the series of teeth 264 of the actuator 256 to be released from the series of teeth 260 of the lock 252 in the noted manner, and thereafter the torque or biasing force exerted by the resilient element 244 to spread the gripping portions 224, 228 apart may be used to provide an aspiration stroke of the plunger 108. This arrangement may limit the syringe 100 from pulling back the plunger 108 unintentionally when, for instance, the gripping portion 320 of the actuator 256 is accidentally or unintentionally depressed. In one arrangement, the user may be required to maintain depression of the gripping portion 320 of the actuator 256 in the direction 360 to allow the resilient element 244 to spread the gripping portions 224, 228 apart once the series of teeth 264 of the actuator 256 are cleared from the series of teeth 260 of the lock 252. More specifically, the user may be required to maintain depression of the gripping portion 320 of the actuator 256 when the resilient element 328 is designed to force the series of teeth 264 back into engagement with the series of teeth 260 which would otherwise disallow further spreading apart of the gripping portions 224, 228 and thus further fluid aspiration.

If additional fluid discharge is desired at step 428 of the method 400 presented in FIG. 3, the method 400 may return back to step 404 for repetition in accordance with the foregoing. Otherwise, the method 400 may end at 432. It should be appreciated that the method 400 shown in FIG. 3 and as discussed above has only been provided to assist the reader in understanding functionalities of the hand-held injector 10. Thus, it is to be understood that at least some of the steps of the method 400 may be performed in an order other than as described. Furthermore, additional or alternative steps may be included without departing from the scope of the present disclosure.

In one variation of the foregoing, a user may disallow or at least limit the actuator 256 from engaging with the lock 252 (e.g., from ratcheting along the series of teeth 260 of the lock 252) during at least part of the discharge stroke of the plunger 108. This may be done by moving the actuator 256 in conjunction or at least substantial unison with the second handle member 212 as the first and second handle members 204, 212 are moved toward one another (e.g., via a squeezing or compressive motion). This may be characterized as a common actuation motion which both: 1) moves the actuator 256 to a disengaged position relative to the lock 252; and 2) operates the handle assembly 200 in a manner that moves the plunger 108 in a discharge direction. Further in this regard and in relation to the view shown in FIG. 1, a user may manually move the actuator 256 counterclockwise about the pivoting joint 312 (coinciding with fourth pivot axis 316), and may also move the gripping portions 224, 228 of the handle members 204, 212 toward one another by pivoting the handle members 204, 212 relative to one another about the pivot member 268 (coinciding with third axis 220, that is parallel to fourth pivot axis 316) by moving the second handle member 212 in a counterclockwise direction about the pivot member 268.

A user of the injector 10 may initially disengage the actuator 256 from the lock 252 by way of squeezing or moving both the second handle member 212 and the actuator 256 towards the first handle member 204 (by moving the actuator 256 relative to the second handle member 212) using a common actuation motion or force. This arrangement may allow a user to obtain a substantially smooth injection action that is free of ratcheting of the series of teeth 264 of the actuator 256 along the series of teeth 260 of the lock 252 while the gripping portions 224, 228 are moved toward one another in the above-noted manner. For instance, from the position of the handle assembly 200 shown in FIG. 5, the user may insert a finger into the finger aperture 324 of the second handle member 212 and simultaneously squeeze both the gripping portion 320 of the actuator 256 and the second handle member 212 towards the first handle member 204. With reference to FIG. 6, squeezing or depressing the gripping portion 320 of the actuator 256 in the direction 360 pivots the series of teeth 264 of the actuator 256 away from the series of teeth 260 of the lock 252 to prevent or at least limit the series of teeth 260, 264 from interlocking as the first and second handle members 204, 212 are squeezed or otherwise moved together. Upon termination of the desired discharge stroke (where the plunger 108 may be moved any desired amount in the discharge direction), the user may either disallow the aspiration stroke by releasing the gripping portion 320 of the actuator 256 (which allows the resilient element 328 to force the series of teeth 264 of the actuator 256 into engagement with the series of teeth 260 of the lock 252 by moving the actuator 256 relative to the second handle member 212), or allow the resilient element 244 to cause an aspiration stroke of the plunger 108 by releasing at least one of the gripping portions 224, 228 while maintaining depression of the gripping portion 320 of the actuator 256 so that the series of teeth 264 of the actuator 256 are cleared from the series of teeth 260 of the lock 252. In one arrangement, any appropriate catch or other feature may be associated with the second handle member 212 or actuator 256, and may be selectively manipulable by a user to maintain bias of the actuator 256 into the position shown in FIG. 6 (e.g., so that resilient element 244 may spread apart the gripping portions 224, 228 and cause a corresponding movement of the plunger 108 in the aspiration direction after a user has terminated the squeezing of the gripping portions 224, 228 and released at least one of the gripping portions 224, 228 (or at least reduced the actuation force used for the discharge operation)).

In summation, the hand-held injector 10 may provide a discharge stroke for the plunger 108 in two different configurations. In one configuration, the actuator 256 only moves a small amount relative to the second handle member 212 as the gripping portions 224, 228 of the handle members 204, 212 are moved at least generally toward one another. This small amount of movement is in effect an oscillation of the actuator 256 about the pivoting joint 312 through a small angle as the teeth 264 of the actuator 256 “ride” along the teeth 260 of the lock 252 and then move into the space between each adjacent pair of teeth 260. That is, the ratchet (collectively the lock 252 and actuator 256) is in an operational state at this time during the discharge stroke in this configuration. The actuator 256 is engaged with the lock 252 at this time.

A discharge stroke for the plunger 108 may also be undertaken with the ratchet (collectively the lock 252 and the actuator 256) in a deactivated state or condition—where the teeth 264 of the actuator 256 have been moved away from the teeth 260 of the lock 252. A user may pivot the actuator 256 counterclockwise about the pivoting joint 312 in the view shown in FIG. 1 to disengage the actuator 256 from the lock 252. This may be done prior to initiating a discharge stroke, concurrently with the start of a discharge stroke, or actually during a discharge stroke. Again, a discharge stroke for the plunger 108 is provided by moving the gripping portions 224, 228 of the handle members 204, 212 at least generally toward one another (which in turn reduces the spacing between the barrel connection portion 226 and the plunger connection portion 230, to in turn move the plunger 108 in a discharge direction).

If the actuator 256 was engaged with the lock 252 during a discharge stroke for the plunger 108, and the actuation force being exerted on one or more of the handle members 204, 212 is at some time reduced or eliminated (e.g., by a user releasing one or both of the handle members 204, 212), the interaction between the actuator 256 and the lock 252 should preclude a complete execution of an aspiration stroke for the plunger 108. Again, the plunger 108 could move a relatively small distance away from the discharge tip 124 before its movement in an aspiration direction is terminated by one or more teeth 264 of the actuator 256 becoming seated within the space between an adjacent pair of teeth 260 of the lock 252. A desired discharge could also be completed with the actuator 256 being disengaged from the lock 252 in the above-noted manner (by a user moving the actuator 256 relative to the second handle member 212), and thereafter the actuator 256 could be released by the user to engage the actuator 256 with the lock 252 (by a movement of the actuator 256 relative to the second handle member 212) while the user maintains the handle members 204, 212 in a fixed position relative to one another (e.g., prior to releasing one or more of the handle members 204, 212). Again, the interaction between the actuator 256 and the lock 252 should preclude a complete execution of an aspiration stroke for the plunger 108, although the plunger 108 may move a very small distance away from the discharge tip 124 before its movement in an aspiration direction is terminated by one or more teeth 264 of the actuator 256 becoming seated within the space between an adjacent pair of teeth 260 of the lock 252.

When an aspiration stroke for the plunger 108 is desired, a user may move the actuator 256 relative to the second handle member 212 (e.g., about the pivoting joint 312) to disengage the actuator 256 from the lock 252—to move the teeth 264 of the actuator 256 away from and out of engagement with the teeth 260 of the lock 252. Again, the hand-held injector 10 may be configured to require at least a slight compression of the handle members 204, 212 (e.g., a force that would tend to move the gripping portions 224, 228 of the handle members 204, 212 toward one another) to take the pressure off of the engaged teeth 264, 260 prior to moving the actuator 256 relative to the second handle member 212 and into a disengaged position relative to the lock 252.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A hand-held injector comprising:
a barrel comprising a discharge nozzle;
a plunger movable relative to the barrel and comprising a head disposed within the barrel;
a first handle comprising a first free end portion and a pivot, wherein the first handle is interconnected with the barrel;
a second handle pivotally coupled to the first handle by the pivot, wherein the second handle is interconnected with the plunger, wherein the second handle comprises a first free end portion, and wherein the first and second handles are configured to provide a discharge stroke when the first free end portions of the first and second handles are moved towards each other to move the plunger in the direction of the discharge nozzle, and to provide an aspiration stroke when the first free end portions of the first and second handles are moved away from each other to move the plunger away from the discharge nozzle;
a self-activating lock mounted on the pivot; and
a selectively operable actuator movably connected to the second handle, wherein the actuator comprises a gripping portion, wherein the self-activating lock is configured to limit the first free end portions of the first and second handles from being moved apart when the self-activating lock is engaged by the actuator, and-wherein the actuator is operable to disengage the self-activating lock when the gripping portion of the actuator is moved relative to the second handle and in a direction towards the first handle, and wherein the gripping portion of the actuator is movable in the direction of the first handle to disengage the self-activating lock without moving the first free end portion of the second handle member in the direction of the first free end portion of the first handle member.

2-11. (canceled)

12. A hand-powered injector comprising:
a barrel comprising a discharge nozzle;
a plunger movable relative to the barrel and comprising a head disposed within the barrel;
a first handle comprising a first free end portion, wherein the first handle is interconnected with the barrel;
a second handle comprising a first free end portion that is disposed in the direction of the discharge nozzle relative to the first free end portion of the first handle when the first free end portions are at a maximum spacing, wherein the second handle is interconnected with the plunger, wherein the second handle is pivotally connected to the first handle at a locking hinge, and wherein the locking hinge comprises:
a stationary gear associated with and maintained in a fixed position relative to the first handle; and
a triggered actuator movably associated with the second handle, wherein the actuator comprises a gripping portion engageable by an operator of the hand-held injector, wherein the triggered actuator is operable to engage with the stationary gear to limit the first free end portions of the first and second handles from moving apart when the triggered actuator is left in an initial position, and-wherein the triggered actuator is movable to a disengaged position relative to the stationary gear by moving the gripping portion of the triggered actuator relative to the second handle and in the direction of the first handle to allow the first free end portions of the first and second handles to move apart, and wherein the triggered actuator is movable to the disengaged position relative to the stationary gear by moving the gripping portion of the triggered actuator relative to the second handle and in the direction of the first handle to allow the first free end portion of the second handle to be moved in the direction of the first free end portion of the first handle to provide a discharge stroke without the triggered actuator being engaged with the stationary gear.

13. (canceled)

14. The hand-powered injector of claim 12, wherein the triggered actuator is pivotal relative to each of the first and second handles and wherein the triggered actuator is pivotally attached to the second handle.

15. (canceled)

16. The hand-powered injector of claim 12, wherein the triggered actuator is operable to move from a first engaged position with the stationary gear to a second engaged position with the stationary gear as the first free end portions of the first and second handles are moved towards each other.

17. The hand-powered injector of claim 16, wherein the triggered actuator is 1 operable to ratchet along the stationary gear between the first and second engaged positions.

18. The hand-powered injector of claim 16, wherein the triggered actuator is operable to be free of contact with the stationary gear while moving between the first and second engaged positions.

19. The hand-powered injector of claim 12, wherein the stationary gear comprises a series of teeth, and wherein the triggered actuator comprises at least one tooth that is adapted to engage with the series of teeth of the stationary gear.

20. The hand-powered injector of claim 12, further comprising:
a biasing member that is adapted to exert a force for biasing the first free end portions of the first and second handles apart.

21. The hand-powered injector of claim 20, wherein the force is operable to bias the triggered actuator and stationary gear into engagement with each other to limit the first free end portions of the first and second handles from being moved apart.

22. (canceled)

23. The hand-powered injector of claim 12, further comprising a resilient element for biasing the triggered actuator towards the stationary gear.

24. (canceled)

25. A hand-actuated fluid delivery device comprising:
a barrel comprising a discharge nozzle;
a plunger that is movable relative to said barrel and comprising a head disposed within said barrel; and
a handle assembly interconnected with and operable to move said plunger relative to said barrel in both a discharge direction and an aspiration direction, wherein said handle assembly comprises:
a first handle member comprising a free end portion, a barrel connection portion, and a pivot;
a second handle member mounted on said pivot and comprising a free end portion and a plunger connection portion, wherein said free end portion of said second handle member is disposed in the direction of said discharge nozzle relative to said free end portion of said first handle member when said free end portions are at a maximum spacing, wherein said first and second handle members are pivotally connected about a first axis of said pivot at a location on said first handle member that is between its said free end portion and its said barrel connection portion and at a location on said second handle member that is between its said free end portion and its said plunger connection portion, wherein said first and second handle members are configured to move said plunger in said discharge direction when said free end portions of said first and second handle members are moved toward each other and which reduces the spacing between said barrel connection portion and said plunger connection portion, and wherein said first and second handle members are configured to move said plunger in said aspiration direction when said free end portions of said first and second handle members are moved away from each other and which increases the spacing between said barrel connection portion and said plunger connection portion;
a plunger lock mounted on said pivot of said first handle member; and
an actuator pivotally connected with said second handle member about a second axis that is spaced from said first axis, wherein said actuator is pivotable about said second axis into an engaged position with said plunger lock to limit said free end portions of said first and second handle members from being moved apart, wherein said actuator is manually pivotable by a user about said second axis into a disengaged position relative to said plunger lock to allow said free end portions of said first and second handle members to be moved apart, and wherein said second axis moves about said first axis when said free end portions of said first and second handle members are moved toward each other while said actuator remains in said engaged position with said plunger lock.

26. The hand-actuated fluid delivery device of claim 25, wherein said handle assembly further comprises at least one biasing member to bias said free end portions of said first and second handle members apart.

27. The hand-actuated fluid delivery device of claim 25, wherein said handle assembly further comprises a ratchet, which in turn comprises said plunger lock and said actuator.

28. The hand-actuated fluid delivery device of claim 25, wherein said plunger lock is maintained in a fixed position relative to said first handle member.

29. The hand-actuated fluid delivery device of claim 25, wherein said plunger lock comprises a gear which in turn comprises a plurality of teeth that are disposed on a perimeter of said gear.

30. The hand-actuated fluid delivery device of claim 25, wherein in a first side view of said hand-actuated fluid delivery device: 1) said second handle member moves counterclockwise about said first axis when said first and second handle members are operated to move said plunger in said discharge direction; and 2) said actuator also moves counterclockwise about said second axis when said actuator is moved into said disengaged position.

31. (canceled)

32. The hand-actuated fluid delivery device of claim 25, wherein said handle assembly further comprises at least one biasing member to bias said actuator into said engaged position.

33. The hand-actuated fluid delivery device of claim 25, wherein said actuator is independently movable relative to said second handle member.

34. The hand-actuated fluid delivery device of claim 25, wherein said actuator is movable from said engaged position to said disengaged position and said first and second handle members are operable to move said plunger in said discharge direction by a common actuation motion.

35. The hand-actuated fluid delivery device of claim 25, wherein said first pivot axis and said second pivot axis are at least substantially parallel with one another.
